(19) United States
(12) Patent Application Publication
(10) Pub. No.: US 2012/0271078 A1
(43) Pub. Date: Oct. 25, 2012

(54) Dehydrogenation Process

(75) Inventors: Tan-Jen Chen, Kingwood, TX (US); Terry E. Helton, Bethlehem, PA (US); Teng Xu, Houston, TX (US)

(73) Assignee: ExxonMobil Chemical Patents Inc., Baytown, TX (US)

(21) Appl. No.: 13/512,805

(22) PCT Filed: Dec. 17, 2010
(86) PCT No.: PCT/US10/61041

§ 371 Date: Jun. 22, 2012

Related U.S. Application Data
(60) Provisional application No. 61/301,799, filed on Feb. 5, 2010.
(60) Provisional application No. 61/334,781, filed on May 14, 2010.

(51) Int. Cl. C07C 5/42

(52) U.S. Cl. 585/252; 585/430; 585/434; 585/433; 585/254

(57) ABSTRACT

In a dehydrogenation process a hydrocarbon stream comprising at least one non-aromatic six-membered ring compound and at least one five-membered ring compound is contacted with a dehydrogenation catalyst comprising: (i) a support; (ii) a first component comprising at least one metal component selected from Group 1 and Group 2 of the Periodic Table of Elements; and (iii) a second component comprising at least one metal component selected from Groups 6 to 10 of the Periodic Table of Elements, wherein the catalyst composition exhibits an oxygen chemisorption of greater than 50%. The contacting is conducted under conditions effective to convert at least a portion of the at least one non-aromatic six-membered ring compound in the hydrocarbon stream to benzene and to convert at least a portion of the at least one five-membered ring compound in the hydrocarbon stream to paraffins.

### PRIORITY CLAIM

This application claims priority to U.S. Provisional Application Ser. No. 61/301,799 filed Feb. 5, 2010; and U.S. Provisional Application Ser. No. 61/334,781 filed May 14, 2010, the disclosures of which are fully incorporated herein by their reference.

### CROSS REFERENCE TO RELATED APPLICATIONS

This patent application is related to U.S. Provisional Application Ser. No. 61/334,767, filed May 14, 2010; U.S. Provisional Application Ser. No. 61/334,775, filed May 14, 2010; U.S. Provisional Application Ser. No. 61/334,784, filed May 14, 2010; and U.S. Provisional Application Ser. No. 61/334,787, filed May 14, 2010, the disclosures of which are fully incorporated herein by their reference.

### FIELD

The present invention relates to a process for dehydrogenating hydrocarbon streams and in particular the C₆-rich streams produced in the hydroalkylation of benzene to produce cyclohexylbenzene.

### BACKGROUND

Various dehydrogenation processes have been proposed to dehydrogenate non-aromatic six membered ring compounds. These dehydrogenation processes are typically used to convert non-aromatic compounds such as cyclohexane into aromatic compounds such as benzene wherein the aromatic compound produced may be used as a raw material in a subsequent process. Alternatively, the aromatic compound produced may be used as a raw material in the same process which produced the non-aromatic compound to be dehydrogenated. For example, the dehydrogenation of cyclohexane to benzene can be important in the hydroalkylation process for producing cyclohexylbenzene as illustrated below.

Cyclohexylbenzene can be produced from benzene by the process of hydroalkylation or reductive alkylation. In this process, benzene is heated with hydrogen in the presence of a catalyst such that the benzene undergoes partial hydrogenation to produce a reaction intermediate such as cyclohexene which then alkylates the benzene starting material. Thus, U.S. Pat. Nos. 4,094,918 and 4,177,165 disclose hydroalkylation of aromatic hydrocarbons over catalysts which comprise nickel- and rare earth-treated zeolites and a palladium promoter. Similarly, U.S. Pat. Nos. 4,122,125 and 4,206,082 disclose the use of ruthenium and nickel compounds supported on rare earth-treated zeolites as aromatic hydroalkylation catalysts. The zeolites employed in these prior art processes are zeolites X and Y. In addition, U.S. Pat. No. 5,053,571 proposes the use of ruthenium and nickel supported on zeolite beta as the aromatic hydroalkylation catalyst. However, these earlier proposals for the hydroalkylation of benzene suffered from the problems that the selectivity to cyclohexylbenzene was low, particularly at economically viable benzene conversion rates, and that large quantities of unwanted by-products, particularly cyclohexane and methylcyclopentane, were produced.

More recently, U.S. Pat. No. 6,037,513 has disclosed that cyclohexylbenzene selectivity in the hydroalkylation of benzene can be improved by contacting the benzene and hydrogen with a bifunctional catalyst comprising at least one hydrogenation metal and a molecular sieve of the MCM-22 family. The hydrogenation metal is preferably selected from palladium, ruthenium, nickel, cobalt and mixtures thereof, and the contacting step is conducted at a temperature of about 50 to 350° C., a pressure of about 100 to 7000 kPa, a benzene to hydrogen molar ratio of about 0.01 to 100 and a weight hourly space velocity (WHSV) of about 0.01 to 100 hr⁻¹. The '513 patent discloses that the resultant cyclohexylbenzene can then be oxidized to the corresponding hydroperoxide and the peroxide decomposed to the desired phenol and cyclohexanone.

Not only does production of impurities such as cyclohexane and methylcyclopentane represent loss of valuable benzene feed, but also overall benzene conversion rates are typically only 40 to 60 wt % so that it is generally necessary to recycle the unreacted benzene. Unless removed, these impurities will tend to build up in the recycle stream thereby displacing benzene and increasing the production of undesirable by-products. Thus, a significant problem facing the commercial application of cyclohexylbenzene as a phenol precursor is removing the cyclohexane and methylcyclopentane impurities in the benzene recycle streams.

One solution to this problem is proposed in U.S. Pat. No. 7,579,511 which describes a process for making cyclohexylbenzene in which benzene undergoes hydroalkylation in the presence of a first catalyst to form a first effluent stream containing cyclohexylbenzene, cyclohexane, methyl cyclopentane, and unreacted benzene. The first effluent stream is then separated into a cyclohexane/methylcyclopentane-rich stream, a benzene-rich stream, and a cyclohexylbenzene-rich stream and the cyclohexane/methylcyclopentane-rich stream is contacted with a second, low acidity, dehydrogenation catalyst to convert at least a portion of the cyclohexane to benzene and at least a portion of the methylcyclopentane to linear and/or branched paraffins and form a second effluent stream. The benzene-rich stream and the second effluent stream can then be recycled to the hydroalkylation step. However, one problem with this process is that cyclohexane and methylcyclopentane have similar boiling points to that of benzene so that their separation by conventional distillation is difficult.

Another solution is proposed in International Patent Publication No. WO2009/131769, in which benzene undergoes hydroalkylation in the presence of a first catalyst to produce a first effluent stream containing cyclohexylbenzene, cyclohexane, and unreacted benzene. The first effluent stream is then divided into a cyclohexylbenzene-rich stream and a C₆ product stream comprising cyclohexane and benzene. At least part of the C₆ product stream is then contacted with a second catalyst under dehydrogenation conditions to convert at least part of the cyclohexane to benzene and produce a second effluent stream which comprises benzene and hydrogen and which can be recycled to the hydroalkylation step.

Both of the processes disclosed in U.S. Pat. No. 7,579,511 and WO2009/131769 rely on the use of a dehydrogenation catalyst comprising a Group VIII metal on a porous inorganic support such as aluminum oxide, silicon oxide, titanium oxide, zirconium oxide, activated carbon and combinations thereof. However, in practice, such a dehydrogenation catalyst has only limited activity for the conversion of methylcyclopentane and in some instances can undergo rapid aging. There is therefore, a need for an improved catalyst for removing cyclohexane and methylcyclopentane from the benzene recycle streams employed in benzene hydroalkylation processes.

According to the present invention, it has now been found that catalyst containing at least one dehydrogenation metal and a Group 1 or Group 2 metal promoter (i.e., alkali metal or alkaline earth metals) are effective catalysts for the dehydrogenation of cyclohexane to benzene and methylcyclopentane to linear and/or branched paraffins in benzene-containing and other hydrocarbon streams in that they exhibit high activity for the conversion of both five- and six-membered non-aromatic rings, and yet have a relatively low aging rate.

### SUMMARY

In one aspect, the invention resides in a dehydrogenation process comprising:
(a) providing a hydrocarbon stream comprising at least one non-aromatic six-membered ring compound and at least one five-membered ring compound; and
(b) producing a dehydrogenation reaction product stream comprising the step of contacting at least a portion of the hydrocarbon stream with a dehydrogenation catalyst, and the contacting being conducted under conditions effective to convert at least a portion of the at least one non-aromatic six-membered ring compound in the hydrocarbon stream to benzene and to convert at least a portion of the at least one five-membered ring compound in the hydrocarbon stream to at least one paraffin;
wherein the dehydrogenation catalyst comprises: (i) a support; (ii) a first component comprising at least one metal component selected from Group 1 and Group 2 of the Periodic Table of Elements wherein the first component is present in an amount of at least 0.1 wt %; and (iii) a second component comprising at least one metal component selected from Groups 6 to 10 of the Periodic Table of Elements and wherein the catalyst composition has an oxygen chemisorption of greater than 50%.

Conveniently, the catalyst composition exhibits an oxygen chemisorption of greater than 55%, such as greater than 60%, such as greater than 65%, and such as greater than 70%.

Conveniently, the support is selected from the group consisting of silica, a silicate, an aluminosilicate, alumina, zirconia, carbon, and carbon nanotubes, and preferably comprises silica.

In one embodiment, the first component comprises at least one metal component selected from potassium, cesium, and rubidium.

Conveniently, the dehydrogenation catalyst has an alpha value from about 0 to about 20, about 0 to about 5, and about 0 to about 1.

Conveniently, the conditions in the contacting (b) comprise a temperature between about 200° C. and about 550° C. and a pressure between about 100 and about 7,000 kPaa.

In one embodiment, the hydrocarbon stream is a C₆ hydrocarbon-rich stream containing benzene, cyclohexane, and methylcyclopentane.

Conveniently, the C₆ hydrocarbon-rich stream is produced by:
(c) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and unreacted benzene; and
(d) separating at least a portion of the hydroalkylation reaction product stream into the C₆ hydrocarbon-rich stream and a cyclohexylbenzene-rich stream.

In another aspect, the invention resides in a process for producing cyclohexylbenzene, the process comprising:
(a) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and unreacted benzene;
(b) separating at least a portion of the hydroalkylation reaction product stream into (i) a C₆-rich stream comprising benzene, cyclohexane, and methylcyclopentane; and (ii) a cyclohexylbenzene-rich stream;
(c) contacting at least a portion of the C₆-rich stream with a dehydrogenation catalyst, the contacting being conducted under conditions effective to convert at least a portion of the cyclohexane to benzene and at least a portion of the methylcyclopentane to at least one paraffin and form a dehydrogenation reaction product stream wherein the dehydrogenation catalyst comprises: (i) a support; (ii) a first component comprising at least one metal component selected from Group 1 and Group 2 of the Periodic Table of Elements wherein the first component is present in an amount of at least 0.1 wt %; and (iii) a second component comprising at least one metal component selected from Groups 6 to 10 of the Periodic Table of Elements and wherein the catalyst composition has an oxygen chemisorption of greater than 50%;
(d) separating at least a portion of the dehydrogenation reaction product stream produced into a C₆ recycle stream and a paraffin-rich stream;
(e) recycling at least a portion of the C₆ recycle stream to the contacting step (a); and
(f) recovering cyclohexylbenzene from the cyclohexylbenzene-rich stream.

Conveniently, the hydroalkylation conditions include a temperature between about 100° C. and about 400° C. and a pressure between about 100 and about 7,000 kPa.

Conveniently, wherein the hydrogen and benzene are fed to the contacting (a) in a molar ratio of hydrogen to benzene of between about 0.15:1 and about 15:1.

Conveniently, hydrogen and benzene are fed to the contacting (a) in a molar ratio of hydrogen to benzene of between about 0.15:1 and about 15:1.

Conveniently, the hydroalkylation catalyst comprises a molecular sieve of the MCM-22 family and a hydrogenation metal.

### DETAILED DESCRIPTION

Described herein is a process for dehydrogenating a hydrocarbon stream comprising at least one non-aromatic six-membered ring compound and at least one non-aromatic five-membered ring compound and optionally at least one aromatic compound, such as benzene. The process comprises contacting at least a portion of the hydrocarbon stream with a dehydrogenation catalyst under conditions effective to convert at least a portion of the at least one non-aromatic six-membered ring compound in the hydrocarbon stream to benzene and to convert at least a portion of the at least one five-membered ring compound in the hydrocarbon stream to at least one paraffin and form a dehydrogenation reaction product stream.

In one embodiment, the hydrocarbon stream comprises at least 10 wt % benzene, at least 20 wt % benzene, at least 30 wt % benzene, at least 40 wt % benzene, at least 50 wt % benzene, at least 60 wt % benzene, at least 70 wt % benzene, and at least 80 wt % benzene. In another embodiment, the hydrocarbon stream comprises at least 1 wt % cyclohexane, at least 5 wt % cyclohexane, at least 10 wt % cyclohexane, and at least 20 wt % cyclohexane. In still another embodiment, the hydrocarbon stream comprises at least 0.05 wt % methylcyclopentane, at least 0.1 wt % methylcyclopentane, and 0.2 wt % methylcyclopentane.

The novel catalyst employed in the dehydrogenation reaction comprises: (i) a support; (ii) a first component; and (iii) a second component produced such that the catalyst exhibits an oxygen chemisorption of greater than 50%, preferably greater than 55%, and more preferably greater than 60%. In another embodiment, the oxygen chemisorption can also be greater than 65%, greater than 70%, and greater than 75%.

Conveniently, the support employed in the dehydrogenation catalyst is selected from the group consisting of silica, alumina, a silicate, an aluminosilicate, zirconia, carbon, and carbon nanotubes, and preferably comprises silica. Impurities which can be present in the catalyst support (e.g., silica) are, for example, sodium salts such as sodium silicate which can be present from anywhere from 0.01 to 2 wt %.

In one embodiment, the dehydrogenation catalyst comprises a silica support having pore volumes and median pore diameters determined by the method of mercury intrusion porosimetry described by ASTM Standard Test D4284. The silica support may have surface areas as measured by ASTM D3663. In one embodiment, the pore volumes are in the range of from about 0.2 cc/gram to about 3.0 cc/gram. The median pore diameters are in the range from about 10 angstroms to about 2000 angstroms or from 20 angstroms to 500 angstroms; and the surface areas (m2/gram) are in the range from 10 to 1000 m2/gram or from 20 to 500 m2/gram. The support may or may not comprise a binder.

Generally, the catalyst comprises a first component comprising at least one metal component selected from Group 1 and Group 2 of the Periodic Table of Elements, such that the first component may comprise any combination or mixture of metal components selected from

Groups 1 and 2 of the Periodic Table of Elements. Typically, the first component is present in an amount of at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, and at least 1.0 wt %. In one embodiment, the first component comprises at least one metal component selected from Group 1 of the Periodic Table of Elements, such as potassium, cesium, and rubidium; preferably potassium and potassium compounds. In another embodiment, the first component comprises at least one metal component selected from Group 1 of the Periodic Table of Elements. In still another embodiment, the first component comprises at least one metal component selected from Group 2 of the Periodic Table of Elements such as beryllium, calcium, magnesium, strontium, barium, and radium; preferably calcium and magnesium. Typically, the first component is present in an amount between about 0.1 and about 5 wt % of the catalyst or between about 0.2 and about 4 wt % of the catalyst or between about 0.3 and about 3 wt % of the catalyst.

In addition, the catalyst comprises a second component comprising at least one metal component selected from Groups 6 to 10 of the Periodic Table of Elements, such as platinum and palladium such that the second component may comprise any combination or mixture of metal components selected from Groups 6 to 10 of the Periodic Table of Elements. In another embodiment, the second component comprises at least one metal component selected from Group 10 of the Periodic Table of Elements.

Typically, the second component is present in an amount between about 0.1 and about 10 wt % of the catalyst such as between about 0.1 and about 5 wt % of the catalyst or between about 0.2 and about 4 wt % of the catalyst or between about 0.3 and about 3 wt % of the catalyst. In another embodiment, the first component is present in an amount of at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, and at least 1.0 wt %.

The term “metal component” is used herein to include elemental metal and a metal compound that may not be purely the elemental metal, but could, for example, be at least partly in another form, such as an oxide, hydride or sulfide form. The weight % (wt %) of the metal component is herein defined as being measured as the metal present based on the total weight of the catalyst composition irrespective of the form in which the metal component is present.

In one embodiment, the dehydrogenation catalyst is produced by initially treating the support, such as by impregnation, with a solution of the first component, such as an aqueous solution of potassium carbonate. After drying, the treated support is calcined, normally in an oxygen-containing atmosphere, such as air, at a temperature of about 100° C. to about 700° C. for a time of about 0.5 to about 50 hours. The calcined support is then treated, again typically by impregnation, with a solution of the second component or a precursor thereof

Optionally, the second component may be impregnated into the support with the aid of at least one organic dispersant. The organic dispersant may help to increase the metal dispersion of the first component. The at least one organic dispersant may be used to increase the metal dispersion of the second component with or without the impregnation of the first component into the support. The at least one organic dispersant is selected from an amino alcohol and an amino acid, such as arginine. Generally, the organic dispersant is present in an amount between about 1 and about 50 wt % of the catalyst support, such as between about 1 and 20 wt % of the catalyst support.

After treatment with the second component, the support is again dried and calcined, normally in an oxygen-containing atmosphere, such as air, at a temperature of about 100° C. to about 600° C. for a time of about 0.5 to about 50 hours.

In an alternative embodiment, the dehydrogenation catalyst is produced by initially treating the support, such as by impregnation, with a solution containing both the first component and the second component or a precursor thereof, optionally together with at least one organic dispersant selected from an amino alcohol and an amino acid, such as arginine. In this case, after drying, a single calcination procedure, normally in an oxygen-containing atmosphere, such as air, at a temperature of about 100° C. to about 700° C. for a time of about 0.5 to about 50 hours, is used to produce the finished catalyst.

After application of each of the first component and second component to the support, the support is preferably heated at a temperature of about 100° C. to about 700° C., for example about 200° C. to about 500° C., such as about 300° C. to about 450° C., for a time of about 0.5 to about 50 hours, such as about 1 to about 10 hours. In addition to removing any liquid carrier and dispersant used to apply the metal component(s) to the support, the heating is believed to assist in bonding the metal to the support and thereby improve the stability of the final catalyst. The heating is preferably conducted in an oxidizing atmosphere, such as air, although a reducing atmosphere, such as hydrogen, can also be employed.

Preferably, the temperature of the calcination after treatment with the first and second component is from about 100° C. to about 600° C.; from about 150° C. to about 550° C.; from about 200° C. to about 500° C., from about 250° C. to about 450° C., and from about 275° C. to about 425° C. In other embodiments, the calcination temperature lower limit may be about 100° C., about 150° C., about 200° C., about 225° C., about 250° C., about 275° C., about 300° C., and about 325° C.; and the upper limit temperature may be about 600° C., about 550° C., about 500° C., about 475° C., about 450° C., about 425° C., about 400° C., about 375° C., and about 350° C. with ranges from any lower limit to any upper limit being contemplated. Preferably, the calcination period is for a time of about 0.5 to about 50 hours.

Preferably, the majority of the calcination after treatment with the first and second component occurs from about 100° C. to about 600° C.; from about 150° C. to about 550° C.; from about 200° C. to about 500° C., from about 250° C. to about 450° C., and from about 275° C. to about 425° C. In other embodiments, the calcination temperature lower limit wherein the majority of the calcination occurs may be about 100° C., about 150° C., about 200° C., about 225° C., about 250° C., about 275° C., about 300° C., and about 325° C.; and the upper limit temperature may be about 600° C., about 550° C., about 500° C., about 475° C., about 450° C., about 425° C., about 400° C., about 375° C., and about 350° C. with ranges from any lower limit to any upper limit being contemplated. Preferably, the calcination period is for a time of about 0.5 to about 50 hours.

Suitable conditions for the dehydrogenation step include a temperature of about 250° C. to about 750° C., a pressure of about atmospheric to about 500 psi-gauge (psig) [100 to 3447 kPa-gauge (kPag)], a weight hourly space velocity of about 0.2 to 50 hr⁻¹, and a hydrogen to hydrocarbon feed molar ratio of about 0 to about 20, such as about 1 to about 5.

Preferably, the temperature of the dehydrogenation process is from about 300° C. to about 750° C.; from about 350° C. to about 650° C.; from about 400° C. to about 550° C.; from about 450° C. to about 550° C.; and from about 400° C. to about 500° C. In other embodiments, the temperature lower limit may be about 350° C.; about 400° C.; about 430° C.; about 440° C.; about 450° C.; about 460° C.; about 470° C.; about 480° C.; and about 490° C.; and the upper limit temperature may be about 500° C.; about 510° C.; about 520° C.; about 530° C.; about 540° C.; about 550° C.; about 600° C.; about 650° C.; about 700° C.; and about 750° C. with ranges from any lower limit to any upper limit being contemplated. In still other embodiments, the temperature lower limit may be about 500° C.; about 510° C.; about 520° C.; about 530° C.; about 540° C.; and about 550° C.; and the upper limit temperature may be about 560° C.; about 570° C.; about 580° C.; about 590° C.; about 600° C.; about 650° C.; about 700° C.; and about 750° C. with ranges from any lower limit to any upper limit being contemplated.

Preferably, the pressure of the dehydrogenation process is from 0 to about 300 psig (0 to 2068 kPag), 50 to 300 psig (345 to 2068 kPag), from 60 to 300 psig (414 to 2068 kPag), from 70 to 300 psig (482 to 2068 kPag), from 80 to 300 psig (552 to 2068 kPag), from 90 to 300 psig (621 to 2068 kPag), and from 100 to 300 psig (689 to 2068 kPag). In other embodiments, the temperature lower limit may be 50 psig (345 kPag), 60 psig (414 kPag), 70 psig (482 kPag), 80 psig (552 kPag), 90 psig (621 kPa), and 100 psig (689 kPag); and the upper limit temperature may be 125 psig (862 kPag), 150 psig (1034 kPag), 175 psig (1207 kPag), 200 psig (1379 kPag), 250 psig (1724 kPag), 300 psig (2068 kPag), 400 psig (2758 kPag), and 500 psig (3447 kPag) with ranges from any lower limit to any upper limit being contemplated. In still other embodiments, the temperature lower limit may be 150 psig (1034 kPag), 160 psig (1103 kPag), 170 psig (1172 kPag), 180 psig (1241 kPag), 190 psig (1310 kPag), and 200 psig (1379 kPag); and the upper limit temperature may be 250 psig (1724 kPag), 300 psig (2068 kPag), 400 psig (2758 kPag), and 500 psig (3447 kPag) with ranges from any lower limit to any upper limit being contemplated.

The reactor configuration used for the dehydrogenation process generally comprises one or more fixed bed reactors containing a solid catalyst with a dehydrogenation function. Per-pass conversion of cyclohexanone using the present catalyst is greater than 70%, and typically at least 95%. Provision can be made for the endothermic heat of reaction, preferably by multiple adiabatic beds with interstage heat exchangers. The temperature of the reaction stream drops across each catalyst bed, and then is raised by the heat exchangers. Preferably, 3 to 5 beds are used, with a temperature drop of about 30° C. to about 100° C. across each bed. Preferably, the last bed in the series runs at a higher exit temperature than the first bed in the series.

Preferably, the alpha value of the dehydrogenation catalyst is from about 0 to about 10, and from about 0 to about 5, and from about 0 to about 1. In other embodiments, the alpha value lower limit may be about 0.0, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, and about 10; and the upper alpha value limit may be about 200, about 175, about 150, about 125, about 100, about 90, about 80, about 70, about 60, about 50, about 40, about 30, about 20, about 10, about 5, about 1.9, about 1.8, about 1.7, about 1.6, about 1.5, about 1.4, about 1.3, about 1.2, about 1.1, about 1, about 0.9, about 0.8, about 0.7, about 0.6, and about 0.5 with ranges from any lower limit to any upper limit being contemplated.

Although the present process can be used with any hydrocarbon stream comprising at least one non-aromatic six-membered ring compound and at least one non-aromatic five-membered ring compound, the process has particular application as part of an integrated process for the conversion of benzene to phenol. In such an integrated process the benzene is initially converted to cyclohexybenzene by any conventional technique, including alkylation of benzene with cyclohexene in the presence of an acid catalyst, such as zeolite beta or an MCM-22 family molecular sieve, or by oxidative coupling of benzene to biphenyl followed by hydrogenation of the biphenyl. However, in practice, the cyclohexylbenzene is generally produced by contacting the benzene with hydrogen under hydroalkylation conditions in the presence of a hydroalkylation catalyst whereby the benzene undergoes the following reaction (1) to produce cyclohexylbenzene (CHB):

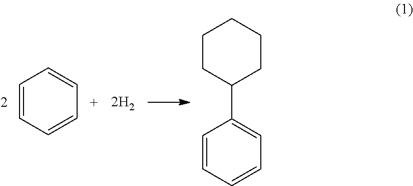

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are between about 100° C. and about 400° C., such as between about 125° C. and about 250° C., while suitable reaction pressures are between about 100 and about 7,000 kPa, such as between about 500 and about 5,000 kPa. Suitable values for the molar ratio of hydrogen to benzene are between about 0.15:1 and about 15:1, such as between about 0.4:1 and about 4:1 for example, between about 0.4 and about 0.9:1.

The catalyst employed in the hydroalkylation reaction is generally a bifunctional catalyst comprising a molecular sieve of the MCM-22 family and a hydrogenation metal. The term “MCM-22 family material” (or “material of the MCM-22 family” or “molecular sieve of the MCM-22 family”), as used herein, includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of MCM-22 family generally have an X-ray diffraction pattern including d-spacing maxima at 12.4±0.25, 6.9±0.15, 3.57±0.07, and 3.42±0.07 Angstrom. The X-ray diffraction data used to characterize the material (b) are obtained by standard techniques using the K-alpha doublet of copper as the incident radiation and a diffractometer equipped with a scintillation counter and associated computer as the collection system. Molecular sieves of MCM-22 family include MCM-22 (described in U.S. Pat. No. 4,954,325); PSH-3 (described in U.S. Pat. No. 4,439,409); SSZ-25 (described in U.S. Pat. No. 4,826,667); ERB-1 (described in European Patent No. 0293032); ITQ-1 (described in U.S. Pat. No 6,077,498); ITQ-2 (described in International Patent Publication No. WO97/17290); MCM-36 (described in U.S. Pat. No. 5,250,277); MCM-49 (described in U.S. Pat. No. 5,236,575); MCM-56 (described in U.S. Pat. No. 5,362,697); UZM-8 (described in U.S. Pat. No. 6,756,030); and mixtures thereof. Preferably, the molecular sieve is selected from (a) MCM-49, (b) MCM-56 and (c) isotypes of MCM-49 and MCM-56, such as ITQ-2.

Any known hydrogenation metal can be employed in the hydroalkylation catalyst, although suitable metals include palladium, ruthenium, nickel, zinc, tin, and cobalt, with palladium being particularly advantageous. Generally, the amount of hydrogenation metal present in the catalyst is between about 0.05 and about 10 wt %, such as between about 0.1 and about 5 wt %, of the catalyst. In one embodiment, where the MCM-22 family molecular sieve is an aluminosilicate, the amount of hydrogenation metal present is such that the molar ratio of the aluminum in the molecular sieve to the hydrogenation metal is from about 1.5 to about 1500, for example, from about 75 to about 750, such as from about 100 to about 300.

The hydrogenation metal may be directly supported on the MCM-22 family molecular sieve by, for example, impregnation or ion exchange. However, in a more preferred embodiment, at least 50 wt %, for example at least 75 wt %, and generally substantially all of the hydrogenation metal is supported on an inorganic oxide separate from, but composited with the molecular sieve. In particular, it is found that by supporting the hydrogenation metal on the inorganic oxide, the activity of the catalyst and its selectivity to cyclohexylbenzene and dicyclohexylbenzene are increased as compared with an equivalent catalyst in which the hydrogenation metal is supported on the molecular sieve.

The inorganic oxide employed in such a composite hydroalkylation catalyst is not narrowly defined provided it is stable and inert under the conditions of the hydroalkylation reaction. Suitable inorganic oxides include oxides of Groups 2, 4, 13, and 14 of the Periodic Table of Elements, such as alumina, titania, and/or zirconia. As used herein, the numbering scheme for the Periodic Table Groups is as disclosed in Chemical and Engineering News, Vol. 63(5), p. 27 (1985).

The hydrogenation metal is deposited on the inorganic oxide, conveniently by impregnation, before the metal-containing inorganic oxide is composited with the molecular sieve. Typically, the catalyst composite is produced by co-pelletization, in which a mixture of the molecular sieve and the metal-containing inorganic oxide are formed into pellets at high pressure (generally about 350 to about 350,000 kPa), or by co-extrusion, in which a slurry of the molecular sieve and the metal-containing inorganic oxide, optionally together with a separate binder, are forced through a die. If necessary, additional hydrogenation metal can subsequently be deposited on the resultant catalyst composite.

Suitable binder materials include synthetic or naturally occurring substances as well as inorganic materials such as clay, silica, and/or metal oxides. The latter may be either naturally occurring or in the form of gelatinous precipitates or gels including mixtures of silica and metal oxides. Naturally occurring clays which can be used as a binder include those of the montmorillonite and kaolin families, which families include the subbentonites and the kaolins, commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment, or chemical modification. Suitable metal oxide binders include silica, alumina, zirconia, titania, silica-alumina, silica-magnesia, silica-zirconia, silica-thoria, silica-beryllia, silica-titania as well as ternary compositions such as silica-alumina-thoria, silica-alumina-zirconia, silica-alumina-magnesia, and silica-magnesia-zirconia.

Although the hydroalkylation step is highly selective towards cyclohexylbenzene, the effluent from the hydroalkylation reaction will normally contain unreacted benzene feed, some dialkylated products, and other by-products, particularly cyclohexane, and methylcyclopentane. In fact, typical selectivities to cyclohexane and methylcyclopentane in the hydroalkylation reaction are 1-25 wt % and 0.1-2 wt % respectively. The hydroalkylation reaction effluent is therefore fed to a separation system normally comprising at least two distillation towers. Given the similar boiling points of benzene, cyclohexane, and methylcyclopentane, it is difficult to separate these materials by distillation. Thus, in a distillation tower, a C₆-rich stream comprising benzene, cyclohexane, and methylcyclopentane is recovered from the hydroalkylation reaction effluent. This C₆-rich stream is then subjected to the dehydrogenation process described above such that at least a portion of the cyclohexane in the stream is converted to benzene and at least a portion of the methylcyclopentane is converted to linear and/or branched paraffins, such as 2-methylpentane, 3-methylpentane, n-hexane, and other hydrocarbon components such as isohexane, C₅ aliphatics, and C₁ to C₄ aliphatics. The dehydrogenation product stream is then fed to a further separation system, typically a further distillation tower, to divide the dehydrogenation product stream into a C₆ recycle stream and a paraffinic stream rich in 2-methylpentane, 3-methylpentane, hexane, and other C₁ to C₆ paraffins. The C₆ recycle stream can then be recycled to the hydroalkylation step, while the paraffinic stream can be used as a fuel for the process.

After separation of the C₆-rich stream, the remainder of hydroalkylation reaction effluent is fed a second distillation tower to separate the monocyclohexylbenzene product from any dicyclohexylbenzene and other heavies. Depending on the amount of dicyclohexylbenzene present in the reaction effluent, it may be desirable to transalkylate the dicyclohexylbenzene with additional benzene to maximize the production of the desired monoalkylated species.

Transalkylation with additional benzene is typically effected in a transalkylation reactor, separate from the hydroalkylation reactor, over a suitable transalkylation catalyst, including large pore molecular sieves such as a molecular sieve of the MCM-22 family, zeolite beta, MCM-68 (see U.S. Pat. No. 6,014,018), zeolite Y, zeolite USY, and mordenite. A large pore molecular sieve has an average pore size in excess of 7 Å in some embodiments or from 7 Å to 12 Å in other embodiments. The transalkylation reaction is typically conducted under at least partial liquid phase conditions, which suitably include a temperature of about 100 to about 300° C., a pressure of about 800 to about 3500 kPa, a weight hourly space velocity of about 1 to about 10 hr⁻¹ on total feed, and a benzene/dicyclohexylbenzene weight ratio about of 1:1 to about 5:1. The transalkylation reaction effluent can then be returned to the second distillation tower to recover the additional monocyclohexylbenzene product produced in the transalkylation reaction.

After separation in the second distillation tower, the cyclohexylbenzene is converted into phenol by a process similar to the Hock process. In this process, the cyclohexylbenzene is initially oxidized to the corresponding hydroperoxide. This is accomplished by introducing an oxygen-containing gas, such as air, into a liquid phase containing the cyclohexylbenzene. Unlike the Hock process, atmospheric air oxidation of cyclohexylbenzene, in the absence of a catalyst, is very slow and hence the oxidation is normally conducted in the presence of a catalyst.

Suitable catalysts for the cyclohexylbenzene oxidation step are the N-hydroxy substituted cyclic imides described in U.S. Pat. No. 6,720,462 and incorporated herein by reference, such as N-hydroxyphthalimide, 4-amino-N-hydroxyphthalimide, 3-amino-N-hydroxyphthalimide, tetrabromo-N-hydroxyphthalimide, tetrachloro-N-hydroxyphthalimide, N-hydroxyhetimide, N-hydroxyhimimide, N-hydroxytrimellitimide, N-hydroxybenzene-1,2,4-tricarboximide, N,N′-dihydroxy(pyromellitic diimide), N,N′-dihydroxy(benzophenone-3,3′,4,4′-tetracarboxylic diimide), N-hydroxymaleimide, pyridine-2,3-dicarboximide, N-hydroxysuccinimide, N-hydroxy(tartaric imide), N-hydroxy-5-norbornene-2,3-dicarboximide, exo-N-hydroxy-7-oxabicyclo[2.2.1]hept-5-ene-2,3-dicarboximide, N-hydroxy-cis-cyclohexane-1,2-dicarboximide, N-hydroxy-cis-4-cyclohexene-1,2 dicarboximide, N-hydroxynaphthalimide sodium salt or N-hydroxy-o-benzenedisulphonimide Preferably, the catalyst is N-hydroxyphthalimide. Another suitable catalyst is N,N′,N″-thihydroxyisocyanuric acid.

These materials can be used either alone or in the presence of a free radical initiator and can be used as liquid-phase, homogeneous catalysts or can be supported on a solid carrier to provide a heterogeneous catalyst. Typically, the N-hydroxy substituted cyclic imide or the N,N′,N″-trihydroxyisocyanuric acid is employed in an amount between 0.0001 wt % to 15 wt %, such as between 0.001 to 5 wt %, of the cyclohexylbenzene.

Suitable conditions for the oxidation step include a temperature between about 70° C. and about 200° C., such as about 90° C. to about 130° C., and a pressure of about 50 to 10,000 kPa. Any oxygen-containing gas, preferably air, can be used as the oxidizing medium. The reaction can take place in batch reactors or continuous flow reactors. A basic buffering agent may be added to react with acidic by-products that may form during the oxidation. In addition, an aqueous phase may be introduced, which can help dissolve basic compounds, such as sodium carbonate.

The final reactive step in the conversion of the cyclohexylbenzene into phenol and cyclohexanone involves cleavage of the cyclohexylbenzene hydroperoxide, which is conveniently effected by contacting the hydroperoxide with a catalyst in the liquid phase at a temperature of about 20° C. to about 150° C., such as about 40° C. to about 120° C., a pressure of about 50 to about 2,500 kPa, such as about 100 to about 1000 kPa. The cyclohexylbenzene hydroperoxide is preferably diluted in an organic solvent inert to the cleavage reaction, such as methyl ethyl ketone, cyclohexanone, phenol or cyclohexylbenzene, to assist in heat removal. The cleavage reaction is conveniently conducted in a catalytic distillation unit.

The catalyst employed in the cleavage step can be a homogeneous catalyst or a heterogeneous catalyst.

Suitable homogeneous cleavage catalysts include sulfuric acid, perchloric acid, phosphoric acid, hydrochloric acid, and p-toluenesulfonic acid. Ferric chloride, boron trifluoride, sulfur dioxide, and sulfur trioxide are also effective homogeneous cleavage catalysts. The preferred homogeneous cleavage catalyst is sulfuric acid, with preferred concentrations in the range of 0.05 to 0.5 wt %. For a homogeneous acid catalyst, a neutralization step preferably follows the cleavage step. Such a neutralization step typically involves contact with a basic component, with subsequent decanting of a salt-enriched aqueous phase.

A suitable heterogeneous catalyst for use in the cleavage of cyclohexylbenzene hydroperoxide includes a smectite clay, such as an acidic montmorillonite silica-alumina clay, as described in U.S. Pat. No. 4,870,217, the entire disclosure of which is incorporated herein by reference.

The effluent from the cleavage reaction comprises phenol and cyclohexanone in substantially equimolar amounts and, depending on demand, the cyclohexanone can be sold or can be dehydrogenated into additional phenol. Any suitable dehydrogenation catalyst can be used in this reaction, such as the dehydrogenation catalyst or a variation of the catalyst described herein. Suitable conditions for the dehydrogenation step comprise a temperature of about 250° C. to about 500° C. and a pressure of about 0.01 atm to about 20 atm (1 kPa to 2000 kPa), such as a temperature of about 300° C. to about 450° C. and a pressure of about 1 atm to about 3 atm (100 kPa to 300 kPa).

Provided are one or more embodiments:
A. A dehydrogenation process comprising:
   (a) providing a hydrocarbon stream comprising at least one non-aromatic six-membered ring compound and at least one five-membered ring compound; and
   (b) producing a dehydrogenation reaction product stream comprising the step of contacting at least a portion of the hydrocarbon stream with a dehydrogenation catalyst under conditions effective to convert at least a portion of the at least one non-aromatic six-membered ring compound to benzene and to convert at least a portion of the at least one five-membered ring compound to at least one paraffin;
   wherein the dehydrogenation catalyst comprises: (i) a support; (ii) a first component comprising at least one metal component selected from Group 1 and Group 2 of the Periodic Table of Elements wherein the first component is present in an amount of at least 0.1 wt %; and (iii) a second component comprising at least one metal component selected from Groups 6 to 10 of the Periodic Table of Elements and wherein the catalyst composition has an oxygen chemisorption of greater than 50%.
B. The process of embodiment A, wherein the dehydrogenation catalyst has an oxygen chemisorption of greater than 60%.
C. The process of any one of embodiments A to B, wherein the dehydrogenation catalyst has an oxygen chemisorption of greater than 65%.
D. The process of any one of embodiments A to C, wherein the dehydrogenation catalyst has an alpha value of less than 10.
E. The process of any one of embodiments A to D, wherein the dehydrogenation catalyst has an alpha value of less than 5.
F. The process of any one of embodiments A to E, wherein the dehydrogenation catalyst has an alpha value of less than 1.
G. The process of any one of embodiments A to F, wherein the support is selected from the group consisting of silica, alumina, a silicate, an aluminosilicate, zirconia, carbon, and carbon nanotubes.
H. The process of any one of embodiments A to G, wherein the support comprises silica.
I. The process of any one of embodiments A to H, wherein the second component comprises at least one metal component selected from platinum and palladium.
J. The process of any one of embodiments A to I, wherein the first component comprises at least one metal component selected from potassium, cesium, and rubidium.
K. The process of any one of embodiments A to J, wherein the first component comprises at least one metal component comprising potassium.
L. The process of any one of embodiments A to K, wherein the conditions in the contacting step (b) comprise a temperature between about 200° C. and about 550° C. and a pressure between about 100 and about 7,000 kPaa.
M. The dehydrogenation process of any one of embodiments A to L, wherein the dehydrogenation catalyst is produced by a method comprising:
   (i) treating the support with the first component;
   (ii) calcining the treated support at a temperature of about 100° C. to about 700° C.;
   (iii) impregnating the support with the second component; and
   (iv) calcining the impregnated support at a temperature of about 100° C. to about 700° C.,
   wherein the impregnating step (iii) is effected prior to or at the same time as the treating step (i).
N. The process of embodiment M, wherein the impregnating step (iii) is affected after the treating step (i) and the calcining step (ii).
O. The process of any one of embodiments M to N, wherein the calcining step (iv) is conducted in an oxygen-containing atmosphere at a temperature of about 200° C. to about 500° C. for a time of about 1 to about 10 hours.
P. The process of any one of embodiments M to O, wherein the calcining step (iv) is conducted in an oxygen-containing atmosphere at a temperature of about 300° C. to about 450° C. for a time of about 1 to about 10 hours.
Q. The process of any one of embodiments M to P, wherein the hydrocarbon stream is a C₆-rich stream comprising at least 50 wt % benzene, at least 5 wt % cyclohexane, and at least 0.1 wt % methylcyclopentane.
R. The process of embodiment Q, wherein the C₆-rich stream is produced by:
   (c) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and benzene; and
   (d) separating at least a portion of the hydroalkylation reaction product stream into the C₆-rich stream and a cyclohexylbenzene-rich stream.
S. The process of embodiment R, and further comprising:
   (e) separating at least a portion of the dehydrogenation reaction product stream produced in the contacting step (b) into a benzene recycle stream and a stream comprising 2-methylpentane and 3-methylpentane; and
   (f) recycling at least a portion of the benzene recycle stream to the contacting step (c).
T. A process for producing cyclohexylbenzene, the process comprising:
   (a) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and benzene;
   (b) separating at least a portion of the hydroalkylation reaction product stream into (i) a C₆-rich stream comprising benzene, cyclohexane, and methylcyclopentane; and (ii) a cyclohexylbenzene-rich stream;
   (c) producing a dehydrogenation reaction product stream comprising the step of contacting at least a portion of the C₆-rich stream with a dehydrogenation catalyst the contacting being conducted under conditions effective to convert at least a portion of the cyclohexane to benzene and at least a portion of the methylcyclopentane to at least one paraffin wherein the dehydrogenation catalyst comprises: (i) a support; (ii) a first component comprising at least one metal component selected from Group 1 and Group 2 of the Periodic Table of Elements, wherein the first component is present in an amount of at least 0.1 wt %; and (iii) a second component comprising at least one metal component selected from Groups 6 to 10 of the Periodic Table of Elements, and wherein the catalyst composition has an oxygen chemisorption of greater than 50%;
   (d) separating at least a portion of the dehydrogenation reaction product stream produced into a benzene recycle stream and a stream comprising 2-methylpentane, 3-methylpentane, and other C₁ to C₆ paraffins;
   (e) recycling at least a portion of the benzene recycle stream to the contacting step (a); and
   (f) recovering cyclohexylbenzene from the cyclohexylbenzene-rich stream.
U. The process of embodiment T, wherein the dehydrogenation catalyst has an oxygen chemisorption of greater than 60%.
V. The process of any one of embodiments T to U, wherein the dehydrogenation catalyst is produced by a method comprising:
   (i) treating the support with the first component;
   (ii) calcining the treated support at a temperature of about 100° C. to about 700° C.;
   (iii) impregnating the support with the second component; and
   (iv) calcining the impregnated support at a temperature of about 100° C. to about 700° C.,
   wherein the impregnating step (iii) is effected prior to or at the same time as the treating step (i).
W. The process of any one of embodiments T to V, wherein the hydroalkylation conditions in the contacting (a) include a temperature between about 100° C. and about 400° C. and a pressure between about 100 and about 7,000 kPa.
X. The process of any one of embodiments T to W, wherein the hydroalkylation catalyst comprises a molecular sieve of the MCM-22 family and a hydrogenation metal.
Y. The process of any one of embodiments T to X, wherein the conditions in the producing step (c) comprise a temperature between about 200° C. and about 550° C. and a pressure between about 100 and about 7,000 kPaa.

When a stream is described as being “rich” in a specified species, it is meant that the specified species in that stream is enriched relative to other species in the same stream or composition on a weight percentage basis. For illustration purposes only, a cyclohexylbenzene-rich stream will have a cyclohexylbenzene wt % greater than any other species or component in that same stream. A “C₆” species generally means any species containing 6 carbon atoms.

As used herein, the oxygen chemisorption value of a particular catalyst is a measure of metal dispersion on the catalyst and is defined as [the ratio of the number of moles of atomic oxygen sorbed by the catalyst to the number of moles of dehydrogenation metal contained by the catalyst] X 100%. The oxygen chemisorption values referred to herein are measured using the following technique.

Oxygen chemisorption measurements are obtained using the Micrometrics ASAP 2010. Approximately 0.3 to 0.5 grams of catalyst are into the Micrometrics. Under flowing helium, the catalyst is ramped from ambient to 250° C. at a rate of 10° C. per minute and held for 5 minutes. After 5 minutes, the sample is placed under vacuum at 250° C. for 30 minutes. After 30 minutes of vacuum, the sample is cooled to 35° C. at 20° C. per minute and held for 5 minutes. The oxygen isotherm is collected in increments at 35° C. between 0.50 and 760 mm Hg.

The invention will now be more particularly described with reference to the following non-limiting Examples and the accompanying drawings.

### EXAMPLE 1

### (Sample A): Preparation of 1 wt % Pt/1 wt % K Silica Catalyst

A platinum/potassium/silica catalyst (Sample A) was prepared by the following procedure. A silica extrudate was impregnated using aqueous based incipient wetness impregnation with 1 wt % K as potassium carbonate followed by air calcination at 540° C. After the potassium impregnation and calcination, a platinum containing 1/20″ (1.3 mm) quadralobe silica extrudate was prepared using tetra-ammine Pt nitrate (1 wt % Pt) solution using aqueous based incipient wetness impregnation. After impregnation, the extrudate was calcined in air at 350° C. The alpha activity of Sample A is essentially negligible i.e., alpha value less than 1.0. The oxygen chemisorption was measured at 70%.

### EXAMPLE 2

### (Sample B): Preparation of 1 wt % Pt/1 wt % K Alumina Catalyst

The Pt/K/Al₂O₃ catalyst (Sample B) was prepared by depositing a commercial Pt/Al₂O₃ catalyst with the desired amount of potassium. The alpha activity of Sample B is essentially negligible i.e., alpha value less than 1.0.

### EXAMPLE 3

### Sample A and Sample B Performance @420° C.

The extrudate catalyst of Sample A was cut into particles of L/D=1 (length/diameter). 250 mg of catalyst was then mixed with 250 mg of 40 mesh quartz chips, and the mixture was packed into a ¼″ (0.64 cm) stainless steel reactor. A liquid mixture of methylcyclopentane, cyclohexane and benzene was delivered to the reactor using an ISCO pump. The liquid feed was vaporized prior to mixing with H₂. The mixture (H₂ and vaporized feed) was fed into the downflow reactor. The reaction was typically run at 500° C. and 100 psig (689 kPag) total reactor pressure, 10 hr⁻¹ WHSV (based on total liquid feed) with a H₂/liquid feed ratio of 2. The liquid feed composition was 1 wt % methylcyclopentane (MCP), 10 wt % cyclohexane (CH), and 89 wt % benzene (Bz).

Prior to the introduction of the liquid feed, the catalyst was pretreated in 50 sccm H₂ at 100 psig (791 kPa) by ramping reactor temperature from room temperature to 420° C. at 2° C./min; the reactor temperature was held at 420° C. for 2 hours under the same H₂ flow and pressure to reduce the platinum on the catalyst to the metallic state.

The effluent from the reactor was sampled using a Valco sampling valve, and the sample was sent to an on-line GC equipped with a FID detector for analysis. All hydrocarbons were quantified and the results were normalized to 100%. H₂ was not included in the analysis. Conversion of methylcyclopentane (MCP) and cyclohexane (CH) was calculated using the following formulae:

MCP conversion in wt %=(wt % of MCP in the feed−wt % of MCP in effluent)/(wt % of MCP in the feed)*100, and

CH conversion in wt %=(wt % of CH in the feed−wt % of CH in effluent)/ (wt % of CH in the feed)*100.

Selectivity was calculated by normalizing all the products to 100 wt % measured in the reactor effluent excluding methylcyclopentane, cylohexane, and benzene. The selectivity data is reported as %.

The performance testing was operated at 420° C., 10 hr⁻¹ WHSV, 2/1 H₂/feed molar ratio, and 100 psig (689 kpag). The cyclohexane conversion started at about 60% on fresh catalyst for Sample A and at about 70% on fresh catalyst for Sample B. Cyclohexane selectivity to benzene for both Sample A and B was approximately 95 to 98%.

The major products from the reaction of methylcyclopentane were mostly 2-methylpentane, 3-methylpentane and hexane, and C₁-C₄, C₅ and heavies. Most of the products are readily separable from benzene via simple distillation. C₁₋₄, C₅ and heavies refer to hydrocarbons that have 1 to 4 carbons, five carbons, and hydrocarbons containing over 6 carbons, respectively. The C₁₋₄ and C₅ are mostly paraffins, while the heavies are mostly substituted benzenes such as xylene and bi-phenyl.

### EXAMPLE 4

### Sample B Stability Performance @460° C.

The stability of Sample B was tested at 460° C., 2 hr⁻¹WHSV, 2/1 H₂/feed ratio, and 50 psig (345 kpag). MCP conversion was close to 20% for at least the initial 50 days time-on-stream. CH conversion stayed above 80% for at least the initial 50 days time-on stream.

### EXAMPLE 5

### 0.5% Pt/1% K/Si02 (Sample C)

A silica extrudate was impregnated using aqueous based incipient wetness impregnation with 1% K as potassium carbonate followed by air calcination at 540° C. After the potassium impregnation and calcination, the sample was impregnated with 0.5 wt % Pt using tetramine Pt nitrate solution using aqueous based incipient wetness impregnation. After impregnation, the extrudate was calcined in air at 250° C. The sample is designated as Sample C. The metal dispersion was measured using a Micromeritics ASAP 2010 Chemisorption Unit. The oxygen chemisorption was 82%.

### EXAMPLE 6

### 0.5% Pt/1% K/SiO2 (Sample D)

A silica extrudate was impregnated using aqueous based incipient wetness impregnation with 1 wt % K as potassium carbonate followed by air calcination at 540° C. After the potassium impregnation and calcination, the sample was impregnated with 0.5 wt % Pt using tetramine Pt nitrate solution using aqueous based incipient wetness impregnation. After impregnation, the extrudate was calcined in air at 350° C. The sample is designated as Sample D. The metal dispersion was measured using a Micromeritics ASAP 2010 Chemisorption Unit. The oxygen chemisorption was 75%.

### EXAMPLE 7

### 0.5% Pt/1% K/SiO2 (Sample E)

A silica extrudate was impregnated using aqueous based incipient wetness impregnation with 1% K as potassium carbonate followed by air calcination at 540° C. After the potassium impregnation and calcination, the sample was impregnated with 0.5 wt % Pt using tetraammine Pt nitrate solution using aqueous based incipient wetness impregnation. After impregnation, the extrudate was calcined in air at 500° C. The sample is designated as Sample E. The metal dispersion was measured using a Micromeritics ASAP 2010 Chemisorption Unit. The oxygen chemisorption was 61%.

After impregnation, the extrudate was dried in air at 121° C. followed by air calcination at 350° C.

### EXAMPLE 8

### 1% Pt on a 1% Ca Silica Extrudate—Calcined 350° C.

A 1% Ca containing 1/20″ (1.3 mm) quadrulobe extrudate was prepared by impregnating a silica extrudate with calcium nitrate (target 1 wt % Ca) using incipient wetness impregnation. After impregnation, the extrudate was dried in air at 121° C. followed by calcination at 538° C. to convert the calcium nitrate to calcium oxide. A 1 wt % Pt containing 1/20″ (1.3 mm) quadrulobe silica extrudate containing 1 wt % Ca was prepared using tetramine platinum hydroxide (target: 1 wt % Pt) solution using aqueous based incipient wetness impregnation. After impregnation, the extrudate was dried in air at 121° C. followed by air calcination at 350° C.

### EXAMPLE 9

### 1% Pt on a 1% Mg Silica Extrudate—Calcined 350° C.

A 1 wt % Mg containing 1/20″ (1.3 mm) quadrulobe extrudate was prepared by impregnating a silica extrudate with magnesium nitrate (target 1 wt % Mg) using incipient wetness impregnation. After impregnation, the extrudate was dried in air at 121° C. followed by calcination at 538° C. to convert the magnesium nitrate to magnesium oxide. A 1 wt % Pt containing 1/20″ (1.3 mm) quadrulobe silica extrudate containing 1 wt % Mg was prepared using tetraammine platinum hydroxide (target: 1 wt % Pt) solution using aqueous based incipient wetness impregnation. After impregnation, the extrudate was dried in air at 121° C. followed by air calcination at 350° C. The oxygen chemisorption was measured at 53%.

### EXAMPLE 10

### 1% K/1% Pt/SiO₂ (Comparative)

1 wt % platinum-containing 1/20″ (1.3 mm) quadrulobe silica extrudate was prepared by incipient wetness impregnation using an aqueous solution of tetramine Pt nitrate. After impregnation, the sample was dried in air at 121° C., and the dried sample designated as 1% Pt/SiO₂. 1 wt % of K was loaded onto 1% Pt/ SiO₂ by incipient wetness impregnation of a potassium carbonate solution. Following potassium impregnation, the sample was dried at 121° C. and then calcined at 350° C. in air for 3 hours. The sample is designated as Sample X. The oxygen chemisorption was measured as 48% which is less than the oxygen chemisorption value of a catalyst prepared by treating with a potassium component first and a platinum component second.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A dehydrogenation process comprising:
(a) providing a hydrocarbon stream comprising at least one non-aromatic six-membered ring compound and at least one five-membered ring compound; and
(b) producing a dehydrogenation reaction product stream comprising the step of contacting at least a portion of the hydrocarbon stream with a dehydrogenation catalyst under conditions effective to convert at least a portion of the at least one non-aromatic six-membered ring compound to benzene and to convert at least a portion of the at least one five-membered ring compound to at least one paraffin;
wherein the dehydrogenation catalyst comprises: (i) a support; (ii) a first component comprising at least one metal component selected from Group 1 and Group 2 of the Periodic Table of Elements wherein the first component is present in an amount of at least 0.1 wt %; and (iii) a second component comprising at least one metal component selected from Groups 6 to 10 of the Periodic Table of Elements and wherein the catalyst composition has an oxygen chemisorption of greater than 50%.

2. The process of claim 1, wherein the dehydrogenation catalyst has an oxygen chemisorption of greater than 60%.

3. The process of claim 1, wherein the dehydrogenation catalyst has an oxygen chemisorption of greater than 65%.

4. The process of claim 1, wherein the dehydrogenation catalyst has an alpha value of less than 10.

5. The process of claim 1, wherein the dehydrogenation catalyst has an alpha value of less than 5.

6. The process of claim 1, wherein the dehydrogenation catalyst has an alpha value of less than 1.

7. The process of claim 1, wherein the support is selected from the group consisting of silica, alumina, a silicate, an aluminosilicate, zirconia, carbon, and carbon nanotubes.

8. The process of claim 1, wherein the support comprises silica.

9. The process of claim 1, wherein the second component comprises at least one metal component selected from platinum and palladium.

10. The process of claim 1, wherein the first component comprises at least one metal component selected from potassium, cesium and rubidium.

11. The process of claim 1, wherein the first component comprises at least one metal component comprising potassium.

12. The process of claim 1, wherein the conditions in the contacting step (b) comprise a temperature between about 200° C. and about 550° C. and a pressure between about 100 and about 7,000 kPaa.

13. The dehydrogenation process of claim 1, wherein the dehydrogenation catalyst is produced by a method comprising:
(i) treating the support with the first component;
(ii) calcining the treated support at a temperature of about 100° C. to about 700° C.;
(iii) impregnating the support with the second component; and
(iv) calcining the impregnated support at a temperature of about 100° C. to about 700° C.,
wherein the impregnating step (iii) is effected prior to or at the same time as the treating step (i).

14. The process of claim 13, wherein the impregnating step (iii) is effected after the calcining step (ii).

15. The process of claim 13, wherein the calcining step (iv) is conducted in an oxygen-containing atmosphere at a temperature of about 200° C. to about 500° C. for a time of about 1 to about 10 hours.

16. The process of claim 13, wherein the calcining step (iv) is conducted in an oxygen-containing atmosphere at a temperature of about 300° C. to about 450° C. for a time of about 1 to about 10 hours.

17. The process of claim 1, wherein the hydrocarbon stream is a C₆-rich stream comprising at least 50 wt % benzene, at least 5 wt % cyclohexane, and at least 0.1 wt % methylcyclopentane.

18. The process of claim 17, wherein the C₆-rich stream is produced by:
(c) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and benzene; and
(d) separating at least a portion of the hydroalkylation reaction product stream into the C₆-rich stream and a cyclohexylbenzene-rich stream.

19. The process of claim 18, and further comprising:
(e) separating at least a portion of the dehydrogenation reaction product stream produced in the contacting step (b) into a benzene recycle stream and a stream comprising 2-methylpentane and 3-methylpentane; and
(f) recycling at least a portion of the benzene recycle stream to the contacting step (c).

20. A process for producing cyclohexylbenzene, the process comprising:
(a) contacting benzene and hydrogen in the presence of a hydroalkylation catalyst under hydroalkylation conditions effective to form a hydroalkylation reaction product stream comprising cyclohexylbenzene, cyclohexane, methyl cyclopentane, and benzene;
(b) separating at least a portion of the hydroalkylation reaction product stream into (i) a C₆-rich stream comprising benzene, cyclohexane, and methylcyclopentane and (ii) a cyclohexylbenzene-rich stream;
(c) producing a dehydrogenation reaction product stream comprising the step of contacting at least a portion of the C₆-rich stream with a dehydrogenation catalyst the contacting being conducted under conditions effective to convert at least a portion of the cyclohexane to benzene and at least a portion of the methylcyclopentane to at least one paraffin wherein the dehydrogenation catalyst comprises: (i) a support; (ii) a first component comprising at least one metal component selected from Group 1 and Group 2 of the Periodic Table of Elements wherein the first component is present in an amount of at least 0.1 wt %; and (iii) a second component comprising at least one metal component selected from Groups 6 to 10 of the Periodic Table of Elements and wherein the catalyst composition has an oxygen chemisorption of greater than 50%;
(d) separating at least a portion of the dehydrogenation reaction product stream produced into a benzene recycle stream and a stream comprising 2-methylpentane, 3-methylpentane and other C₁ to C₆ paraffins;
(e) recycling at least a portion of the benzene recycle stream to the contacting step (a); and
(f) recovering cyclohexylbenzene from the cyclohexylbenzene-rich stream.

21. The process of claim 20, wherein the dehydrogenation catalyst has an oxygen chemisorption of greater than 60%.

22. The process of claim 20, wherein the dehydrogenation catalyst is produced by a method comprising:
(i) treating the support with the first component;
(ii) calcining the treated support at a temperature of about 100° C. to about 700° C.;
(iii) impregnating the support with the second component; and
(iv) calcining the impregnated support at a temperature of about 100° C. to about 700° C.,
wherein the impregnating step (iii) is effected prior to or at the same time as the treating step (i).

23. The process of claim 20, wherein the hydroalkylation conditions in the contacting (a) include a temperature between about 100° C. and about 400° C. and a pressure between about 100 and about 7,000 kPa.

24. The process of claim 20, wherein the hydroalkylation catalyst comprises a molecular sieve of the MCM-22 family and a hydrogenation metal.

25. The process of claim 20, wherein the conditions in the producing step (c) comprise a temperature between about 200° C. and about 550° C. and a pressure between about 100 and about 7,000 kPaa.
